(19) United States
(12) Patent Application Publication
(10) Pub. No.: US 2026/0191477 A1
(43) Pub. Date: Jul. 9, 2026

(54) METHOD FOR CLASSIFICATION OF ANORECTAL MANOMETRY EXAMS DATA

(71) Applicant: DIGESTAID - ARTIFICIAL INTELLIGENCE DEVELOPMENT, LDA., Gondomar (PT)

(72) Inventors: Miguel José DA QUINTA E COSTA DE MASCARENHAS SARAIVA, Porto (PT); João Pedro SOUSA FERREIRA, Valongo (PT); Maria CASTRO PAUPÉRIO VILA POUCA, Valongo (PT); João Pedro LIMA AFONSO, Monção (PT); Tiago Filipe CARNEIRO RIBEIRO, Lordelo (PT); Manuel Guilherme GONÇALVES DE MACEDO, Porto (PT); Hélder Manuel CASAL CARDOSO, Gondomar (PT); Ana Patricia RIBEIRO ANDRADE, Porto (PT)

(21) Appl. No.: 19/131,092

(22) PCT Filed: Sep. 20, 2023
(86) PCT No.: PCT/PT2023/050031

§ 371 Date: May 19, 2025

(30) Foreign Application Priority Data
Nov. 23, 2022 (PT) 118356

(51) Int. Cl. A61B 5/00; G16H 50/30

(52) U.S. Cl. CPC A61B 5/7275; G16H 50/30

(57) ABSTRACT

The present invention relates to a computational method capable of automatically identifying and characterizing abnormalities in data outputs and deviations for the anatomical entities represented in anorectal manometry, differentiating between obstructed defecation and manometric patterns of fecal incontinence, using previously time series pressure signals archived or tabular data to train a deep learning model tailored to classify the newly acquired data.

### BACKGROUND OF THE INVENTION

The present invention relates to the area of medical data analysis for identifying and characterizing abnormalities and deviations to the anatomical entities depicted in the anorectal manometry data. It is also within the scope of the present invention a computational method to train a deep learning model and further usage of such model for identifying such abnormalities in data outputs from pressure sensing devices configured to be inserted into a user.

Such model uses previously archived time-series pressure signals or tabular data to train a deep learning model adapted to classify newly acquired data.

Training classifiers with transformed data of specific domains, such as contour plot images, signal features, and signal properties, are well-known techniques in the field of medicine. Although image classification has been an active area of research in medicine for some time, now, it has become increasingly popular in recent years with the emerging field of Deep Learning for Image and Signal Classification. Many techniques have been proposed and evaluated to classify data, all relying on both domain-or event-specific labeling to achieve competitive accuracies.

Concerning anorectal manometry, Joshua et al. [reference] proposed a video-based Deep-Learning approach to detect Dyssynergic Defecation through 3D High-Definition Anorectal Manometry. Further, Zifan et al. [reference] proposed an Endoflip vs High-Definition Manometry in the Assessment of Fecal Incontinence. Both approaches developed single-domain classifiers of image or time series signals on a particular event of the entire time series data acquired during an examination. Concerning esophageal manometry, Kou et al. published a study using a Deep-learning-based model for identifying different swallow types in esophageal high-resolution manometry, a multi-stage machine learning model for diagnosis of esophageal manometry and a deep-learning-based unsupervised model on esophageal manometry using variational autoencoder combining two classifiers of a particular event, one in the signal domain and the other in the image domain, to form a blended classifier. These works use merely one data domain to train the classifiers. Another further issue of such approaches is the use of a limited set of model architecture to extract features relevant in classifying pressure profiles. This event-specific labeling limits the classifiers to a narrow target domain.

It is worth mentioning that classification methods such as those just mentioned are standard in the literature; however, it is assumed herein that medical exams are a combination of classifiable events in a time series of profile-generating features in signal and image domains. An example is a manometry examination, which produces a sequence of events of 2D pressure signals. Conventionally, the clinician marks the events of interest after the examination, and then analyze each event by traditional methods such as image inspection or calculation on the signal properties. Such methods require expertise and are time consuming to produce a final diagnosis. For the medical community, event detection should be easily accessible (i.e., acquired efficiently and effortlessly) for diagnosing on platforms where best approaches have not yet been proposed, where packages and libraries may not yet be available, and consumers are strange to machine learning.

In the medical diagnosis of featured data, reliable and fast event classification is necessary to provide better patient care services. Furthermore, classifiers must be time-efficient, as a long-lasting maneuver can be uncomfortable for the patient, often preventing its repetition. Hence, classifiers also should be trained as soon as more data is available. Thus, efficient and fast training methods are necessary for model deployment in an open loop. In a scenario where such requirements are not mandatory, most research focuses on one domain or event and scarcely considers a general method that could fit into a broader application. Another further issue in the prior art approaches is that methods are specific to static data. On demand training should be efficient as new exams are likely to produce valuable training data.

Despite years of research, there is yet no efficient methods to select a classifier of medical exams that can incorporate new data features while maintaining acceptable metrics for a reliable diagnosis. Hence, the present invention addresses an efficient training system that runs training sessions in parallel. Data and computing resources are evenly distributed among the available workers. Each classifier among all considered to train is allocated to one worker. Each worker of a training session trains a classifier using training and new data, thus evaluating classifier performance with features that new data might have. Here, classifier training is generally made with less, but still representative, data; Also, classifier training can run with training options that speed up the process [divergent items with the procedure of this invention].

Document WO 2022/061346 discloses a method for classifying the functionality of the upper gastrointestinal tract from probe data. The document discloses the use of specific artificial intelligence algorithms of binary, multiclass, or multilabel classifiers for esophageal measurement data. In such embodiments one can use multiple AI-based classifiers in sequence to reach the classification of the measurement data relevant for the exam diagnosis. Contrarily, the current invention uses method selection, a medical exam classifier among a multitude of available artificial intelligence classifiers. These medical exams include, but not only, anorectal manometry and gastrointestinal endoscopies. These classifiers include, but are not limited to, decision trees, gradient boosting, support vector machines, random forests, and artificial neural networks.

Current approaches require event-specific knowledge to extract features helpful in classifying data profiles. A new applicable invention now meets the unfulfilled need for adaptive training of classifiers. When comparing the present invention to the aforementioned documents, the invention presents the following technical advantages:
Multiple architectures are considered, which improves the features extraction as architectures best fit to the specific data chosen over the less performing architectures;
Strategic data folding and split for each arch ensures that the features of the new data are going to be trained on all possible model architectures;
Adaptable training for dynamic environments may reduce bias and increase generalization with time.

### BRIEF SUMMARY OF THE INVENTION

The current invention relates to a method for training and selecting a classifier of pressure time-series profiles. In one embodiment, it is realized in one or more tangible computer media capable of executing instructions for performing a method of running a program on a computing system, the computing system operating under one or more computational workers, the method including issuing instructions from one or more computational workers to generate a multitude of adaptable trained classifiers.

Additionally or optionally, these early stopping trainings can be executed in parallel.

In this preferred embodiment, the method includes generating classified feature data indicative of anorectal disorders, causing fecal incontinence (FI), obstructed defecation (OD) or other evacuation dysfunctions from a computational system. Such output features are used to assess the sensorimotor function, reflex activity, recto anal coordination and, ultimately, the voluntary and unvoluntary control of anal continence. A multitude of machine learning algorithms can also be trained in such systems using training data to generate classified feature data from the exam measurement. The measurement data is applied to the selected machine learning algorithm, generating an output indicative of the functional state in the subject.

Training data for performing the method hereby described includes patients diagnosed with OD and FI. Low-pass filters are applied to denoise and trim the time-series data from the medical exam into a sequence of events. Features of each event are extracted, these may include energy, entropy, zero Crossing Counts, mean, standard deviation, median, absolute maximum, minimum, skewness, kurtosis and fast fourier transforms. To avoid overfitting, highly correlated features and not correlated features are removed. The resulting dataset comprises the collected signal features along with the target value.

In the preferred embodiment of the present invention, a model adapted to distinguish between patients with manometric findings of OD from those with FI was built. Merely as an example, multiple machine learning models were trained to understand which best learns from the manometry pressure profiles. In particular, k-nearest neighbors (KNN), support vector machines (SVM), random forests (RF), and gradient boosting (xGB) models were considered. A dummy model can be used for baseline comparisons. Further, a stratified n-fold strategy is applied in each model training and repeated for a predefined number of epochs. To improve performance, each model's hyperparameters are fine-tuned using random splits of 90% of the data for training and the remaining 10% for testing. In the preferred embodiment of the present invention, each model's performance in differentiating between manometric evidence of FI and OD is assessed by comparing at least one or a combination of the following: precision, recall (sensitivity), f1-score, or the accuracy. In another embodiment, the discriminating performance of each model was assessed by analyzing receiver operating curves (AUROC). The classification of each model was validated by the medical diagnosis.

The foregoing and other aspects and advantages of the present disclosure will appear from the following description. In this description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 illustrates a method for training medical examination data classifiers, according to an embodiment of the present invention.

FIG. 2 exemplifies the necessary resources for the selection of a model, according to an embodiment of the present invention.

FIG. 3 exemplifies the steps for training the selected model (252).

FIG. 4 Illustrates a possible configuration for the computational system adapted to perform the method of the present invention.

FIG. 5 Illustrates the accuracy of the studied machine learning models where models were trained 10 times using 5-fold splits.

FIG. 6 Illustrates the confusion matrices for the testing dataset for the tuned models.

FIG. 7 Illustrates the summary of the performance of the machine learning models. Models were tuned by training five times randomly selecting 90% of the data for training and 10% for testing.

FIG. 8 illustrates the accuracy (which mean ± standard
deviation) for each tested model, where xGB is gradient boost;
RF is random forests; SVM is Support vector machines and KNN is k-nearest neighbors.

FIG. 9 illustrates the accuracy (mean ± standard deviation) for each tuned model, where xGB is gradient boost, RF is random forests, SVM is Support vector machines and KNN is k-nearest neighbors.

FIG. 10 illustrates Receiver operator characteristics analyses for discriminating between manometric patterns of obstructed defecation and fecal incontinence for each of the tuned models, where xGB is gradient boost, RF is random forests, SVM is Support vector machines, KNN is k-nearest neighbors, TPR is True Positive Rate and FPR is False Positive Rate.

### DETAILED DESCRIPTION

The present invention discloses a method to train classifiers of measured data from medical exams, which includes imaging, probing data from sensors, or tabular data. The method described here discloses the implementation of training machine learning models, or combination thereof, to classify data from a medical examination. As used herein, the term “domain” is the data nature of the classifier algorithm-for example, an image, an N-dimensional time series, signal features, signal properties, etc.

The term “event” corresponds to a specific time interval of interest. “An embodiment” or “another embodiment” of the present disclosure is not to be interpreted as excluding the existence of additional embodiments that also incorporate the recited features.

“Worker” is a specialized computing system that runs a set of predefined specific tasks.

“Open loop” refers to a system that trains models using the data at a storage module receiving data from a server.

“Training data” refers to the source data that has been used for model training.

“New data” refers to the source data which hasn't yet been used in training.

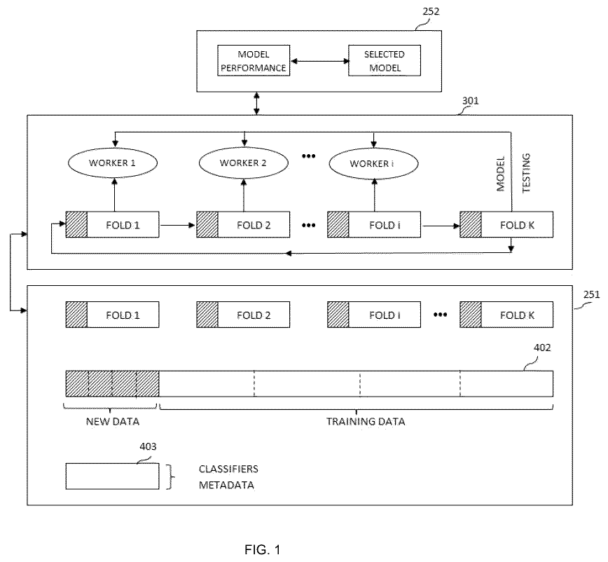

FIG. 1 comprises the method (253) for selecting a ranking model after training from the respective performance metrics (252) on which the selected model was trained and evaluated. Training (301) comprises a series of works in which the different classification models considered are tested. The distribution (251) of data and metadata comprises a method for integrating and distributing medical examination data, wherein the database integrates new data into the training data (402) and the metadata (403) represents a list of architectures, hyperparameters, and training options.

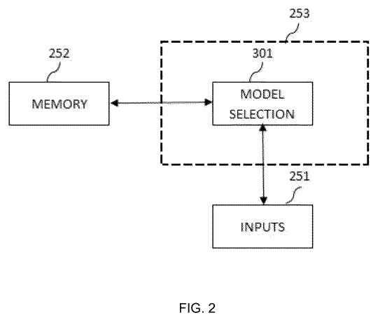

FIG. 2 comprises the training method (253) for selecting the classification model (301) that is stored in memory (252) and the data (251).

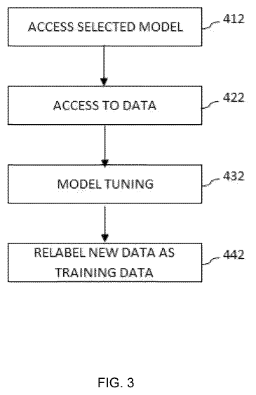

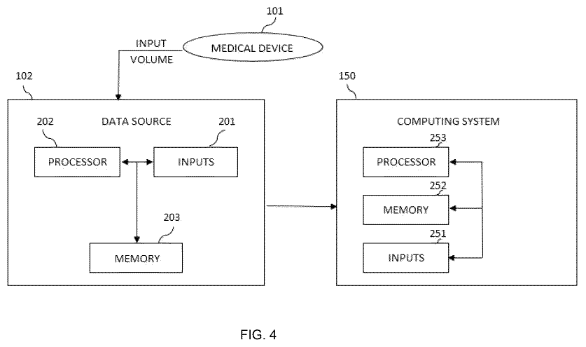

FIG. 3 comprises the steps for training the selected model (412). The selected model (412) is trained by accessing to data (422) in model tuning (432). After tuning, new data is cataloged as training data (442).

The data source 102 receives new data from exam input volumes of a medical device. The medical device is a manometry probe. Additionally, preprocess input data at the processor 202 can be done. A computing system 150 receives one or more types of measured data from the data source 102. The computing system 150 has in memory 252 metadata including, but not solely, a multitude of machine learning architectures, trained machine learning models and a multitude of sets of hyperparameters. In the preferred embodiment, the computing device can execute at least a subset of the preferred classifiers to classify at least a portion of the measured data, or generate feature data from the medical exam. The computing system 150 access training input labeled data 201 and the required metadata in memory 252 to process model selection 300.

The model selection 300 evaluates the output of running k−1 workers. In a further embodiment, each worker is set to run the tasks for training a machine learning algorithm of a specific architecture with model tuning. Training data is split into k folds. Additionally, or alternatively, new data can be copied to each fold. Each fold is then allocated to an available worker until all folds passed through all workers, which ensures the new data will be equally split by all workers, leaving out one fold for testing. The training set is split for model training to generate the data pointers of the all images and ground-truth labels, required to run the model selection. Preferably, patient-specific data is exclusive to one and one fold only to avoid data leakage. Preferably, and to pose sample representativeness, K-fold is applied with stratified grouping by patient in the training set to generate the pointers of the data and ground-truth labels.

Each worker executes a training session for a specific machine learning model architecture. In a preferred embodiment, such training includes hyperparameters tuning, learning rate scheduler, and early stopping. The performance metrics of each run are saved for evaluation of the best-performed model. The selected machine learning model architecture is then trained using the entire training data and ground-truth labels.

A method and system are described for classifying anorectal manometry data acquired from a subject anorectal portion of the gastrointestinal tract with a sensing probe or any other measurement device. The systems and methods described in the present disclosure implement classification algorithms, machine learning algorithms, or combinations thereof, in order to classify these data. For instance, patterns in the input data can be identified and classified using one or more classification and/or machine learning algorithms.

In general, embodiments of the present invention provide AI methodology to classify anorectal measurement data into relevant pathologic groups, including fecal incontinence (FI), obstructed defecation (OD) or other evacuation dysfunctions from a computational system. In these instances, classification algorithms including k-nearest neighbors (KNN), support vector machines (SVM), random forests (RF), and gradient boosting (xGB) models and/or artificial neural networks can be implemented.

A preferred embodiment of the invention discloses a method for anorectal manometry probe data classification which includes the acquisition of exam data from a medical device such as a sensing probe and storage of such data in a computational device or similar with means of data storage. Furthermore, this method preferably cleans the data by means of filtering in order to homogenize the data with the remaining anorectal manometry probe data in the storage. Such stored anorectal manometry probe data has been previously classified as having features indicative of FI/OD or other evacuation system dysfunctions. The method hereby described is provided with means to distinguish between such previously labeled and stored data from newly acquired manometry probe data.

Further, the method of the described invention accesses a computational system, locally or remotely a cloud-based system, whereas a number of workers not inferior to the number of algorithms considered is reserved. Preferably, each worker is then assigned to train labeled data, in parallel with the remaining workers, using the assigned algorithm.

Further and additionally, the method hereby described is provided with means for comparing the performance of each training performed by the workers/data pairings. Optionally, the training data is split in k folds with stratified grouping by patient which keeps grouped data features, thus facilitating feature extraction by the model being trained.

The method ensures new data in all folds with patient split. In some embodiments, where patient split is not possible, i.e., when the number of new patients p is less than k−1: q=(k−1)−p>0, the method randomly distributes the new data among the remaining q folds. Keeping new data in all folds ensures that the features of the new data are going to be trained on all possible model architectures.

The method distributes the k−1 folds to workers, leaving one fold out for model testing. Further, by having a worker training on a fold instead of k−1 folds is faster because we have, still representative, less data for training.

In some embodiments, each training is improved by training options such as early stopping, and learning rate scheduler trigger training end sooner, saving the worker metrics in the storage. Hence the efficiency of the method is improved and the computational resources necessary for the training are optimized.

In some embodiments, the method asynchronously repeats the previously described steps until all folds went through all workers. By having all workers being trained asynchronously and using a minimal representative set of data (a stratified patient-specific fold), data usage and computational system usage are optimized.

Further and additionally, the method evaluates workers metrics and selects the best performing model. By best performing, it should be understood that it can relate to a number of different concepts, namely by comparing them at least with one or a combination of the following: precision, recall (sensitivity), f1-score, or the accuracy. In a further embodiment, the discriminating performance of each model was assessed by analyzing receiver operating curves (AUROC). The classification of each model was validated by the medical diagnosis.

The method of the present invention uses the algorithm considered the best and is assigned to one or a number of workers who can train the algorithm in all the training data and specific training options. Tuning with full data is made only on the classifier that showed greater potential.

Further and additionally, the method is provided with means to access the storage and store the new data labeled as training data for further usage and continuous improvement of the method.

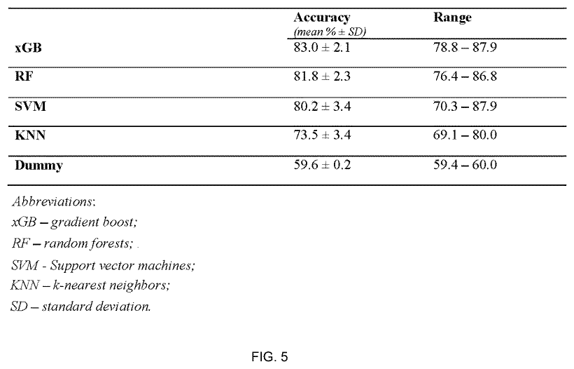

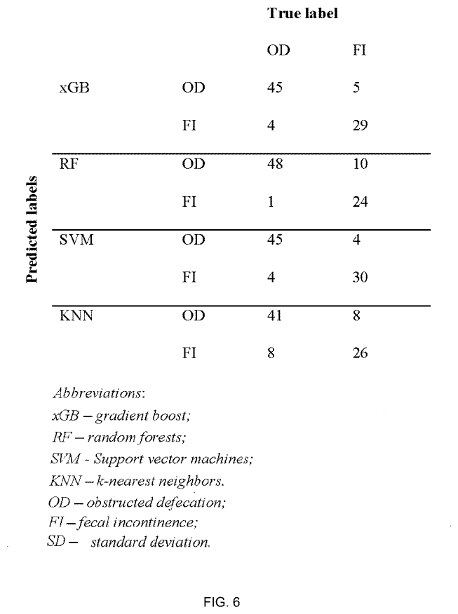

A preferred embodiment of the present invention considers four machine learning models (xGB, RF, SVM, and KNN) to train a total of 827 manometry exams, 493 of which presented a diagnosis of OD, while the remaining 334 were diagnosed with FI. Optionally, five folds were considered, and the accuracy of the four machine learning models is summarized in Table 1 (FIG. 5). Further and optionally, the models'hyperparameters were tuned to achieve maximal performance using random splits of the data in a ratio of 90% for the training dataset (n=744) and 10% for the testing dataset (n=83). The method considers each model and trains on the time-series data, transformed or not, to detect an event. The confusion matrices for the validation dataset of each ML model are shown in Table 2 (FIG. 6) and the accuracies of the tuned models are displayed on FIG. 6.

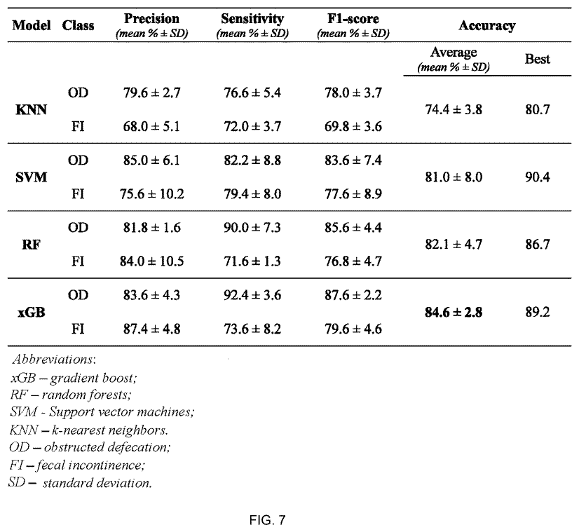

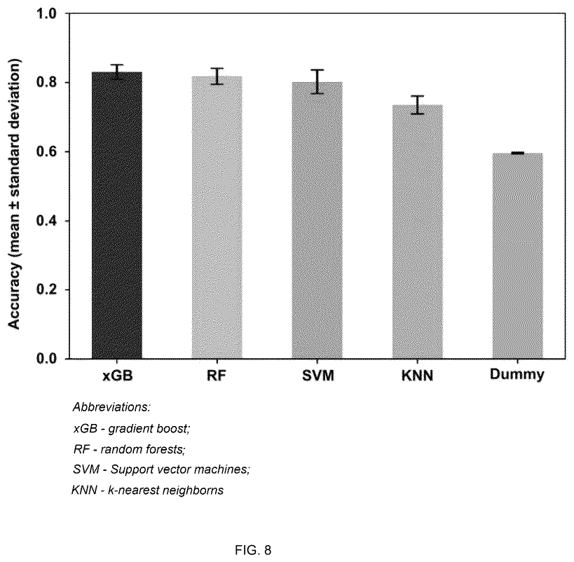

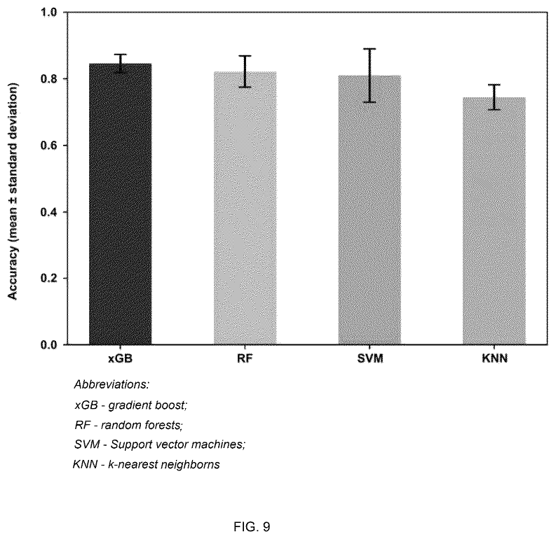

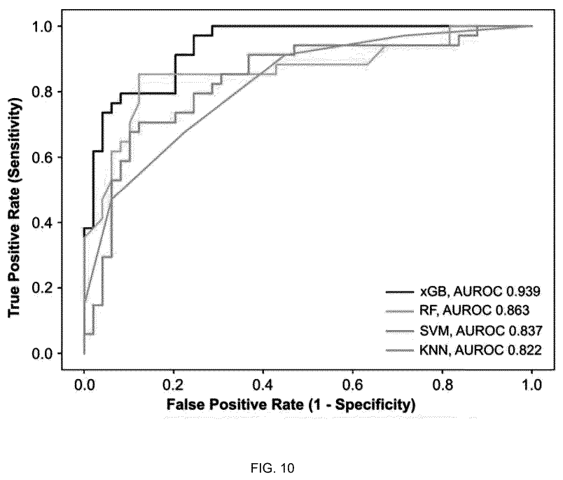

Optionally, the performance marks of the different tuned models, including the precision, sensitivity and the F1-score, were considered and are summarized in Table 3 (FIG. 7). The xGB model showed the highest precision levels for the detection of OD (83.6±4.3%) and FI (87.4±4.8%). The sensitivity for the detection of OD was higher for the xGB machine learning model (92.4±3.65%), similar to that of the RF model (90.0±7.3%). The sensitivity for the detection of FI was similar across the different ML models, with the highest value recorded for SVM (79.4±8.0%). The harmonic mean of precision and sensitivity (F1-score) was higher for the xGB model. Overall, the performance for discriminating between OD and FI was higher for the xGB model, with an AUROC of 0.933.

### REFERENCES

[1] Levy, J., Navas, C. M., Chandra, J. A., Christensen, B., Vaickus, L. J., Curley, M. A., Chey, W. D., Baker, J. R. and Shah, E. D., 2021. Video-Based Deep Learning to Detect Dyssynergic Defecation with 3D High-Definition Anorectal Manometry. bioRxiv.
[2] Zifan, A., Sun, C., Gourcerol, G., Leroi, A. M. and Mittal, R. K., 2018. Endoflip vs high-definition manometry in the assessment of fecal incontinence: a data-driven unsupervised comparison. Neurogastroenterology & Motility, 30(12), p.e13462.
[3] Kou, W., Carlson, D. A., Baumann, A. J., Donnan, E. N., Schauer, J. M., Etemadi, M. and Pandolfino, J. E., 2022. A multi-stage machine learning model for diagnosis of esophageal manometry. Artificial Intelligence in Medicine, 124, p.102233.

1. A computer-implemented method capable of automatically identifying and classifying fecal incontinence and obstructed defecation events in manometry-acquired pressure time-series data, the method comprising:
accessing previously labeled anorectal measurements of pressure data and newly acquired anorectal measurements of pressure data;
equally splitting anorectal measurements of pressure data into a plurality of equally divided folds ensuring each patient data is one and one only fold;
assigning all except one fold with a worker adapted to training a machine learning model and one fold for model testing wherein said training early-stops based on a learning rate scheduler trigger;
asynchronously repeating the previously described steps until all folds went through all workers;
training and validating the full extent of anorectal measurements of pressure data using the best performing model;
testing the newly acquired anorectal measurements of pressure data in the trained model and identifying the existence of pathological events;
predicting fecal incontinence (FI) and obstructed defecation (OD) dysfunctions based on the convolutional features extracted from the anorectal measurements of pressure data.

2. The method of claim 1, wherein the machine learning models are a combination of decision trees, gradient boosting, support vector machines, random forests and convolutional neural networks.

3. The method of claim 1, wherein best performing is assessed by one or a combination of: precision, recall (sensitivity), f1-score, accuracy or the analysis of the receiver operating curves (AUROC).

4. The method of claim 1, wherein the trained machine learning algorithm is trained on the training data in order to identify fecal incontinence or obstructed defecation in the feature map of the manometry time-series pressure data and to generate the classified feature data on the identification of said fecal incontinence or obstructed defecation patterns in the convoluted feature map.

5. The method of claim 2, wherein best performing is assessed by one or a combination of: precision, recall (sensitivity), f1-score, accuracy or the analysis of the receiver operating curves (AUROC).

6. The method of claim 2, wherein the trained machine learning algorithm is trained on the training data in order to identify fecal incontinence or obstructed defecation in the feature map of the manometry time-series pressure data and to generate the classified feature data on the identification of said fecal incontinence or obstructed defecation patterns in the convoluted feature map.

7. The method of claim 3, wherein the trained machine learning algorithm is trained on the training data in order to identify fecal incontinence or obstructed defecation in the feature map of the manometry time-series pressure data and to generate the classified feature data on the identification of said fecal incontinence or obstructed defecation patterns in the convoluted feature map.
